(19) United States
(12) Patent Application Publication
(10) Pub. No.: US 2007/0274942 A1
(43) Pub. Date: Nov. 29, 2007

(54) Aqueous Composition Comprising a Polyionic Dendritic Polymer and an Ionic Surface-Active Agent

(75) Inventors: Mikel Morvan, Princeton (FR); Franck Touraud, rue de la Vallee (FR); Katerina Karagianni, Paris (FR); Jean-Francois Berret, Sevres (FR)

Correspondence Address: Jean-Louis Seugnet;Rhodia INC., 8 Cedar Brook Drive, Cranbury, NJ, 08512-7500, US

(73) Assignee: TOYO SEIKAN KAISHA, LTD., TOKYO JAPAN (JP)

(21) Appl. No.: 10/574,043

(22) PCT Filed: Sep. 29, 2004
(86) PCT No.: PCT/FR04/02462

§ 371 Date: Apr. 23, 2007

Related U.S. Application Data
(60) Provisional application No. 60/506,788, filed on Sep. 29, 2003.
(60) Provisional application No. 60/506,817, filed on Sep. 29, 2003.

(51) Int. Cl. A61K 8/06

(52) U.S. Cl. 424/70.19; 510/119; 510/159; 510/407; 510/417

(57) ABSTRACT

The invention relates to a composition comprising a single-phase aqueous phase consisting of a system containing: water; a water-dispersible or water-soluble polyionic dendritic polymer (a); at least one ionic surface active agent (b); and, optionally, a polycationic or polyanionic polymer (c) which is different from the dendritic polymer. The inventive composition can be used for surface treatments, such as on hair or skin, e.g. for hair conditioning.

A subject matter of the present invention is a composition comprising a monophase aqueous phase comprising a system comprising water, a water-soluble or water-dispersible polyionic dendritic polymer (a), an ionic surfactant (b) and optionally a polycationic or polyanionic polymer (c) other than the dendritic polymer.

The use of polymers for treating surfaces, for example for conditioning the hair or the skin, is known. For example, the use of cationic derivatives of guar or of cellulose or the use of synthetic polymers in shampoos is known.

There exists a need for novel compositions or for novel treatments which can contribute advantageous properties when they are applied to surfaces. There also exist requirements in terms of properties of the compositions in themselves, such as the appearance, the texture, the sensory or cosmetic properties and/or the stability.

The invention meets at least some of these needs by providing a composition comprising:
a monophase aqueous phase comprising a system comprising:
   water,
   a water-soluble or water-dispersible polyionic dendritic polymer (a),
   at least one ionic surfactant (b),
   optionally a polycationic or polyanionic polymer (c) other than the dendritic polymer, and
   optionally at least one amphoteric or zwitterionic or neutral surfactant (d),
in which:
   (a) is polycationic, (b) is anionic and (c), if it is present, is polyanionic or polycationic, or
   (a) is polycationic, (b) is cationic and (c) is present and polyanionic, or
   (a) is polyanionic, (b) is cationic and (c), if it is present, is polyanionic or polycationic, or
   (a) is polyanionic, (b) is anionic and (c) is present and polycationic,
   the monophase aqueous phase becomes two-phase or remains monophase on diluting with water, and
optionally solid or liquid particles of water-insoluble organic or inorganic compounds (e).

For example, the composition can be an aqueous cosmetic composition for the hair and/or skin intended to be rinsed off. It can, for example, be a shampoo, a conditioner or a shower gel. According to one alternative, the composition is a conditioner not intended to be rinsed off.

The invention also provides a process for the treatment of a surface comprising the following stages:
a) application of the composition, and
b) optionally rinsing off.

The invention also provides for the use of the above composition or process in the conditioning, repair or protection of the hair and/or in the dyeing of the hair.

The compositions according to the invention can be cosmetic compositions intended for the treatment of the skin or hair and intended to be applied and rinsed off, such as shampoos or shower gels, exhibit advantageous properties in terms of transparency or deposition of matter (conditioning effect), and/or more generally to optimize cosmetic effects, such as softness, manageability, disentangling, sheen or aptitude for styling, on dry or wet hair. They can protect the individual hair, provide luster, repair cracks in the cortex, reduce negative effects of bleaching and perming, repair the damaged individual hair or protect the color.

In addition, the composition can be used for surface treatments: it can bring about deposition at the surface of at least one of the compounds chosen from (a), (b), (c), if it is present, and (e) if it is present. For example, (a) exhibits a specific affinity for damaged hairs (bleaching operations, ageing, and the like) or damaged areas of the hairs, for example the tips.

The composition can also be used for treatments of surfaces or modifications, for example for rendering textile surfaces or hard surfaces hydrophilic on a more or less lasting basis (for example, resistant to rain and/or to rinsing operations). Rendering hydrophilic can prevent the appearance of marks on drying (marks which may be left by drops of water) and/or facilitate the following cleaning operations and/or prevent the formation of stains and/or dirty marks after cleaning.

In addition, the compositions are easy to prepare, are easy to employ and are satisfactorily stable.

### DEFINITIONS

In the present patent application, the term “monophase aqueous phase” is understood in contrast with a two-phase phase, obtained from the monophase phase, within this phase, for example by diluting. In other words, the phase separation takes place in the aqueous phase and the phase-separated ingredients are ingredients of the system which were not ingredients of the system in the monophase aqueous phase. Thus, the composition can comprise solid or liquid particles of water-insoluble organic or inorganic compounds (e) dispersed in the monophase aqueous phase but which, in the present patent application, are not regarded as forming part of the monophase or two-phase aqueous phase. The two-phase phase can be marked by the appearance of macroscopic or microscopic objects, forming a macroscopic phase separation which can separate on settling, or forming a haze in the aqueous phase, for example in the form of complexes or colloids. This may be observed visually and/or using a microscope and/or by light-scattering or absorption techniques.

The term “dendritic polymer” refers to macromolecular compounds comprising several branchings. They can be regular dendrimers or hyperbranched polymers.

The terms “water-soluble” or “water-dispersible” are to be understood at the pH of the composition and mean that a compound does not form a macroscopic phase separation in water at the pH of the composition at 25° C. at a concentration of greater than 1%.

Monophase Aqueous Phase and Ingredients of the System

The monophase aqueous phase comprises water, (a), (b), optionally (c) and optionally (d).

According to a specific form, (a), (b), (c), if it is present, and (d) if it is present, and their amounts are such that the monophase aqueous phase becomes two-phase on diluting with water, for example at the pH of the composition (pH of the aqueous phase) or at the pH induced by the dilution.

According to a specific form:
the monophase aqueous phase becomes two-phase on diluting with water, and
(a) or (c) precipitates when the aqueous phase becomes two-phase on diluting with water.

According to a specific form, the system comprises (c) and:
(a) is polycationic, (b) is anionic and (c) is polycationic, or
(a) is polyanionic, (b) is cationic and (c) is polyanionic.

The composition can in particular comprise (by weight, with respect to the weight of the composition):
from 0.01 to 10%, preferably from 0.1 to 5%, preferably from 0.1 to 1%, of (a),
from 0.1 to 40, preferably from 1 to 20%, preferably from 5 to 15%, of (b),
from 0% to 20%, preferably from 0 to 5%, preferably from 0 to 3%, of (c),
from 0 to 20% of (d), preferably from 0 to 5%, if (d) is amphoteric or zwitterionic.

Furthermore, the composition can comprise at least 90% by weight of the aqueous phase and the aqueous phase can comprise from 5 to 40% by weight, preferably from 10 to 20% by weight, of the system formed of (a), (b), optionally (c) and optionally (d). The aqueous phase can constitute the whole of the composition.

Further details with regard to ingredients (a), (b), (c) and (d) of the system which it is possible to use are given below.

Dendritic Polymer (a)

The dendritic polymer is polyionic. This means that it comprises cationic or anionic ionic groups at the pH of the composition. These groups can be included at ends of polymer chains or within macromolecular chains. Ionic groups are generally regarded as hydrophilic. It is specified that the term “ionic group” is understood to mean a group which exhibits a charge at any pH or a group which can comprise a charge at a certain pH. In the latter case, reference is sometimes made to potentially ionic groups.

The dendritic polymer can comprise hydrophobic groups and hydrophilic groups. The hydrophobic groups can be included in repeat units within the polymer. They can, for example, be alkylene groups, at least divalent, comprising at least 3 consecutive carbon atoms or groups, at least divalent, comprising a phenyl unit, for example the phenylene group. They are advantageously a group of formula —(CH₂)ₙ— where n is greater than or equal to 3, for example 4, 5, 6 or 11, and/or a group of formula —C₆H₄—.

The hydrophilic groups can be included in repeat units within the polymer and/or be included at the end of the polymer chains. The hydrophilic groups included in repeat units are often regarded as polymerization functional groups. They are, for example, groups or functional groups of formulae —COO— (polyesters), —O— (polyethers), —CONH— (polyamide), —OCOO— (polycarbonate), —NH—COO— (polyurethane), —N< (polyamide), —NH—CO—NH— (urea) or —CO—NH—CO— (imide).

It should be noted that the possibility is not ruled out of ends of polymer chains comprising hydrophobic groups, such as alkyl groups. Neither is the possibility ruled out of ends of polymer chains comprising hydrophilic or hydrophobic nonionic groups. The presence of such groups can help in adjusting the properties of the dendritic polymer.

The dendritic polymer preferably comprises ionic or potentially ionic (depending, for example, on the pH) groups at the ends of the polymer chains. Furthermore, the nature and the properties of these groups can be more easily controlled, modified or varied, either during the polymerization or after, by postfunctionalization.

Examples of ionic groups include:
acid groups, such as sulfonic acid, phosphonic acid or carboxylic acid groups and their sulfonate, phosphate, phosphonate or carboxylate basic forms (anionic groups),
primary, secondary or tertiary amine groups or their ammonium acidic forms, and quaternary ammonium groups (cationic groups).

It should be mentioned that the hydrophilicity and/or the ionic nature of the group may depend on the pH. In the present patent application, the term “hydrophilic group” or “ionic group” respectively denotes groups which are hydrophilic or ionic respectively at any pH and groups having a hydrophilicity or ionic nature respectively which depends on the pH (potentially hydrophilic groups).

Examples of the dendritic polymers include:
dendrimers with a polypropyleneimine backbone, such as the Starburst® range marketed by DSM,
dendrimers with a polyamidoester (or polyesteramide) backbone, such as the Hybrane® range marketed by DSM,
dendrimers with a polyamidoamine backbone (PAMAM),
polyether dendrimers,
diaminobutane-aminopropyl hyperbranched polymers DAB(PA)ₙ,
hyperbranched polyesters, such as the Boltorn® range marketed by Perstorp.

Hyperbranched polyesters and hyperbranched polyamides are in particular dendritic polymers particularly suitable for the implementation of the invention.

According to an advantageous embodiment, the dendritic polymer is a polymer capable of being obtained by a process comprising the following stages:

Stage a) polycondensation of at least one multifunctional monomer of formula (I) comprising at least three reactive polycondensation functional groups,
A-R—(B)_{f} (I)

in which formula
f is an integer greater than or equal to 2, preferably ranging from 2 to 10, very particularly equal to 2,
the symbol A represents a reactive functional group or a group carrying a reactive functional group chosen from the amino, carboxyl, hydroxyl, oxiranyl, halo or isocyanato functional groups or their precursors,
the symbol B represents a reactive functional group or a group carrying a reactive functional group chosen from the amino, carboxyl, hydroxyl, oxiranyl, halo or isocyanato functional groups or their precursors which is an antagonist of A,
the symbol R represents a linear or branched aliphatic, cycloaliphatic or aromatic polyvalent hydrocarbon residue comprising from 1 to 50, preferably from 3 to 20, carbon atoms which is optionally interrupted by one or more oxygen, nitrogen, sulphur or phosphorus heteroatoms, said residue optionally carrying functional groups not capable of reacting with the A and B functional groups,

Stage b) optionally at least partial anionic or cationic ionic functionalization of the polymer obtained in the polycondensation stage.

The symbol B represents a reactive functional group which is an antagonist of the reactive functional group A; this means that the functional group B is capable of reacting with the functional group A by condensation.

Thus, the functional groups which are antagonists
of an amino functional group are in particular the carboxyl (formation of an amide), isocyanato (formation of a urea) or oxiranyl (formation of a β-hydroxylated secondary or tertiary amine) functional groups,
of a carboxyl functional group are in particular the amino (formation of an amide), hydroxyl (formation of an ester) or isocyanato (formation of an amide) functional groups,
of a hydroxyl functional group are in particular the carboxyl (formation of an ester), oxiranyl (formation of an ether) or isocyanato (formation of an amide) functional groups,
of an oxiranyl functional group are in particular the hydroxyl (formation of an ether), carboxyl (formation of an ester) or amino (formation of a β-hydroxylated secondary or tertiary amine) functional groups,
of an isocyanato functional group are in particular the amino, hydroxyl or carboxyl functional groups,
of a halo functional group are in particular the hydroxyl functional groups.

Mention may in particular be made, among amino functional group precursors, of amine salts, such as hydrochlorides.

Mention may in particular be made, among carboxyl functional group precursors, of esters, preferably C₁-C₄, very particularly C₁-C₂, esters, acid halides, anhydrides or amides.

Mention may in particular be made, among hydroxyl functional group precursors, of epoxy compounds.

According to an alternative embodiment, said polycondensation operation is carried out in addition in the presence:
of at least one bifunctional monomer in the linear form of formula (II) or in the corresponding cyclic form comprising two reactive polycondensation/polymerization functional groups
   A′-R′—B′ (II)
in which formula:
   the symbol A′, which is identical to or different from A, represents a reactive functional group chosen from the amino, carboxyl, hydroxyl, oxiranyl, halo or isocyanato functional groups or their precursors which is an antagonist of B and B′,
   the symbol B′, which is identical to or different from B, represents a reactive functional group chosen from the amino, carboxyl, hydroxyl, oxiranyl, halo or isocyanato functional groups or their precursors which is an antagonist of A and A′,
   the symbol R′, which is identical to or different from R, represents a linear or branched aliphatic, cycloaliphatic or aromatic polyvalent hydrocarbon residue comprising from 1 to 50, preferably from 3 to 20, carbon atoms which is optionally interrupted by one or more oxygen, nitrogen, sulphur or phosphorus heteroatoms, said residue optionally carrying functional groups not capable of reacting with the A, A′, B and B′ functional groups,
   the reactive functional group A′ being capable of reacting with the B functional group and/or the B′ functional group by condensation;
   the reactive functional group B′ being capable of reacting with the A functional group and/or the A′ functional group by condensation;
and/or of at least one “core” monomer of formula (III) comprising at least one functional group capable of reacting by condensation with the monomer of formula (I) and/or the monomer of formula (II)
   R¹—(B″)ₙ (III)
in which formula
   n is an integer greater than or equal to 1, preferably ranging from 1 to 100, very particularly from 1 to 20,
   the symbol B″ represents a reactive functional group, identical to or different from B or B′, chosen from the amino, carboxyl, hydroxyl, oxiranyl, halo or isocyanato functional groups or their precursors which is an antagonist of A and A′,
   the symbol R¹ represents a linear or branched aliphatic, cycloaliphatic or aromatic polyvalent hydrocarbon residue comprising from 1 to 50, preferably from 3 to 20, carbon atoms which is optionally interrupted by one or more oxygen, nitrogen, sulphur or phosphorus heteroatoms or an organosiloxane or polyorganosiloxane residue, said R¹ residue optionally carrying functional groups not capable of reacting with the A, A′, B, B′ and B″ functional groups,
   the reactive functional group B″ being capable of reacting with the A functional group and/or the A′ functional group by condensation;
and/or of at least one “chain-limiting” monofunctional monomer of formula (IV)
   A″-R² (IV)
in which formula
   the symbol A″ represents a reactive functional group, identical to or different from A or A′, chosen from the amino, carboxyl, hydroxyl, oxiranyl, halo or isocyanato functional groups or their precursors which is an antagonist of B, B′ and B″,
   the symbol R² represents a linear or branched aliphatic, cycloaliphatic or aromatic polyvalent hydrocarbon residue comprising from 1 to 50, preferably from 3 to 20, carbon atoms which is optionally interrupted by one or more oxygen, nitrogen, sulphur or phosphorus heteroatoms or an organosiloxane or polyorganosiloxane residue, said R² residue optionally carrying functional groups not capable of reacting with the A, A′, A″, B, B′ and B″ functional groups,
   the reactive functional group A″ being capable of reacting with the B functional group and/or the B′ functional group and/or the B″ functional group by condensation;
at least one of the reactive functional groups of at least one of the monomers of formula (II), (III) or (IV) being capable of reacting with an antagonistic functional group of the multifunctional monomer of formula (I).

Preferably, the A, A′, A″ and B, B′, B″ functional groups are chosen from reactive functional groups or groups carrying reactive functional groups chosen from the amino, carboxyl, hydroxyl or oxiranyl functional groups or their precursors. More preferably still, said functional groups are chosen from reactive amino and carboxyl functional groups or groups carrying reactive amino and carboxyl functional groups or their precursors.

For good implementation of the invention:
the molar ratio of the monomer of formula (I) to the monomer of formula (II) is advantageously greater than 0.05 and preferably ranges from 0.125 to 2;
the molar ratio of the monomer of formula (III) to the monomer of formula (I) is advantageously less than or equal to 1, preferably less than or equal to ½ and more preferably still ranges from 0 to ⅓; said ratio ranges very particularly from 0 to ⅕;
the molar ratio of the monomer of formula (IV) to the monomer of formula (I) is advantageously less than or equal to 10, preferably less than or equal to 5; said ratio ranges very particularly from 0 to 2, when f is equal to 2.

The fundamental entity taken into consideration in defining the various molar ratios is the molecule. It is obvious that the expression “condensation reaction” also includes the notion of addition reaction when one or more antagonistic functional groups of at least one of the monomers employed is included in a ring (lactams, lactones or epoxides, for example).

Mention may be made, as examples of monomer (I), of:
5-aminoisophthalic acid,
6-aminoundecanedioic acid,
3-aminopimelic acid,
aspartic acid,
glutamic acid,
3,5-diaminobenzoic acid,
3,4-diaminobenzoic acid,
lysine,
α,α-bis(hydroxymethyl)propionic acid,
α,α-bis(hydroxymethyl)butyric acid,
α,α,α-tris(hydroxymethyl)acetic acid
α,α-bis(hydroxymethyl)valeric acid
α,α-bis(hydroxy)propionic acid
3,5-dihydroxybenzoic acid
or their mixtures.

Mention may be made, as examples of bifunctional monomer of formula (II), of:
ε-captolactam,
aminocaproic acid,
para- or meta-aminobenzoic acid,
11-aminoundecanoic acid,
lauryllactam,
12-aminododecanoic acid,
hydroxyacetic acid (glycolic acid),
hydroxyvaleric acid,
hydroxypropionic acid,
hydroxypivalic acid,
glycolide,
δ-valerolactone,
β-propiolactone,
ε-caprolactone,
lactide
lactic acid
or their mixtures.

More preferably, the bifunctional monomers of formula (II) are the monomers used for the manufacture of linear thermoplastic polyamides. Thus, mention may be made of ω-aminoalkanoic compounds comprising a hydrocarbon chain having from 4 to 12 carbon atoms, or the lactams derived from these amino acids, such as ε-caprolactam. The bifunctional monomer preferred for the implementation of the invention is ε-caprolactam.

According to an advantageous form of the invention, at least a portion of the bifunctional monomers (II) are in the prepolymer form.

Mention may be made, as examples of the monomers (III), of:
aromatic or aliphatic monoamines, such as dodecylamine, octadecylamine, benzylamine, and the like,
aromatic or aliphatic monoacids comprising from 1 to 32 carbon atoms, such as benzoic acid, acetic acid, propionic acid or saturated or unsaturated fatty acids (dodecanoic acid, oleic acid, palmitic acid, stearic acid, and the like),
monofunctional alcohols or epoxides, such as ethylene oxide, epichlorohydrin, and the like,
isocyanates, such as phenyl isocyanate, and the like,
biprimary diamines, preferably linear or branched saturated aliphatic biprimary diamines having from 6 to 36 carbon atoms, such as, for example, hexamethylenediamine, trimethylhexamethylenediamine, tetramethylenediamine or m-xylenediamine,
saturated aliphatic dicarboxylic acids having from 6 to 36 carbon atoms, such as, for example, adipic acid, azelaic acid, sebacic acid, maleic acid or maleic anhydride,
difunctional alcohols or epoxides, such as ethylene glycol, diethylene glycol, pentanediol or glycidyl ethers of monofunctional alcohols comprising from 1 to 24 carbon atoms,
diisocyanates, such as toluene diisocyanates, hexamethylene diisocyanate, phenylene diisocyanate or isophorone diisocyanate,
triols or polyols or aromatic or aliphatic triamines, triacids or polyacids, such as N,N,N-tris(2-aminoethyl)amine, melamine, and the like, citric acid, 1,3,5-benzenetricarboxylic acid, and the like, 2,2,6,6-tetra(β-carboxyethyl)cyclohexanone, trimethylolpropane, glycerol, pentaerythritol or glycidyl ethers of di-, tri- or polyfunctional alcohols,
polymeric compounds, such as the poly- or monoaminated polyoxyalkylenes sold under the Jeffamine® trademark,
aminated polyorganosiloxanes, such as aminated polydimethylsiloxanes.

The preferred “core” monomers (III) are: hexamethylenediamine, adipic acid, Jeffamine® T403, sold by Huntsman, 1,3,5-benzenetricarboxylic acid and 2,2,6,6-tetra(β-carboxyethyl)cyclohexanone.

Mention may be made, as examples of the monomers (IV), of:
aromatic or aliphatic monoamines, such as dodecylamine, octadecylamine or benzylamine. The majority of these compounds are generally regarded as hydrophobic and nonionic.
aromatic or aliphatic monoacids comprising from 1 to 32 carbon atoms, such as benzoic acid, acetic acid, propionic acid or saturated or unsaturated fatty acids (dodecanoic acid, oleic acid, palmitic acid, stearic acid, and the like). The majority of these compounds are generally regarded as hydrophobic and nonionic.
monofunctional alcohols or epoxides, such as ethylene oxide or epichlorohydrin. The majority of these compounds are generally regarded as hydrophobic and nonionic.
isocyanates, such as phenyl isocyanate. The majority of these compounds are generally regarded as hydrophobic and nonionic.
polymeric compounds, such as the monoaminated polyoxyalkylenes for example sold under the Jeffamine M® trademark, such as Jeffamine M 1000® and Jeffamine M 2070®. The majority of these compounds are generally regarded as hydrophilic and nonionic.
monoaminated silicone chains, such as monoaminated polydimethylsiloxanes. The majority of these compounds are generally regarded as hydrophobic and nonionic.
N,N-DiMethylAminoPropylAmine (hydrophilic or potentially hydrophilic, cationic or potentially cationic, as basic or quaternizable, for example with dimethyl sulfate).
N,N-DiEthylAminoPropylAmine (hydrophilic or potentially hydrophilic, cationic or potentially cationic, as basic or quaternizable, for example with dimethyl sulfate).
N,N-DiButylAminoPropylAmine (hydrophilic or potentially hydrophilic, cationic or potentially cationic, as basic or quaternizable, for example with dimethyl sulfate).
N-(3-AminoPropyl)Morpholine (hydrophilic or potentially hydrophilic, cationic or potentially cationic, as basic or quaternizable, for example with dimethyl sulfate).
N-Methyl-N′-(3-AminoPropyl)Piperazine (hydrophilic or potentially hydrophilic, cationic or potentially cationic, as basic or quaternizable, for example with dimethyl sulfate).
N-(3-AminoPropyl)Piperidine (hydrophilic or potentially hydrophilic, cationic or potentially cationic, as basic or quaternizable, for example with dimethyl sulfate).
the mixtures of these compounds.

Mention may in particular be made, among functional groups which may be present in the monomers (I) to (IV) and which are not capable of reacting with the A, A′, A″, B, B′ and B″ functional groups, of functional groups capable of introducing hydrophilicity to, of improving the hydrophilicity of or of conferring an ionic nature on dendritic polymers employed according to the invention. Mention may be made, by way of examples, of the quaternary ammonium (cationic), nitrile (anionic), sulfonate (anionic), phosphonate (anionic), phosphate (anionic), hydroxyl (nonionic), poly(ethylene oxide), ether (nonionic) or tertiary amine (basic or quaternizable, potentially cationic) functional groups.

Mention may in particular be made of:
4-aminobenzenesulfonic acid (anionic) and its ammonium or alkali metal salts, in particular its sodium salt [monomer of formula (II)]
5-sulfosalicylic acid (anionic) [monomer of formula (II)]
D- or L-2-amino-5-phosphonovaleric acid (anionic) [monomer of formula (II)]
sulfobenzoic acid (anionic) and its ammonium or alkali metal salts [monomer of formula (III) or (IV)]
epoxypropyltrimethylammonium chloride (cationic) [monomer of formula (III) or (IV)]
polytioxyl polyethylene glycol (nonionic);
aminomethylphosphonic acid [monomer of formula IV)].

The hydrophilic and/or ionic functional groups can in particular be carried by the monomer (IV), for example by one of the following monomers:
polymeric compounds, such as the monoaminated polyoxyalkylenes for example sold under the Jeffamine M® trademark, such as Jeffamine M 1000® and Jeffamine M 2070®. The majority of these compounds are generally regarded as hydrophilic and nonionic.
N,N-DiMethylAminoPropylAmine (hydrophilic or potentially hydrophilic, cationic or potentially cationic, as basic or quaternizable, for example with dimethyl sulfate).
N,N-DiEthylAminoPropylAmine (hydrophilic or potentially hydrophilic, cationic or potentially cationic, as basic or quaternizable, for example with dimethyl sulfate).
N,N-DiButylAminoPropylAmine (hydrophilic or potentially hydrophilic, cationic or potentially cationic, as basic or quaternizable, for example with dimethyl sulfate).
N-(3-AminoPropyl)Morpholine (hydrophilic or potentially hydrophilic, cationic or potentially cationic, as basic or quaternizable, for example with dimethyl sulfate).
N-Methyl-N′-(3-AminoPropyl)Piperazine (hydrophilic or potentially hydrophilic, cationic or potentially cationic, as basic or quaternizable, for example with dimethyl sulfate).
N-(3-AminoPropyl)Piperidine (hydrophilic or potentially hydrophilic, cationic or potentially cationic, as basic or quaternizable, for example with dimethyl sulfate).

Finally, the possibility is not ruled out of the. dendritic polymer carrying, at the ends of the polymer chains, a mixture of hydrophilic groups and of hydrophobic groups and/or mixtures of ionic and nonionic groups, for example contributed by monomers (IV) and/or acid/base control. It is thus possible to adjust the emulsifying properties and, if appropriate, to render the action of the dendritic polymer sensitive to external conditions.

The dendritic polymers described above can be compared with arborescent structures having a focal point formed by the A functional group and a periphery covered with B endings. It is specified that the fact that the periphery is covered with B endings does not exclude the possibility of B endings being present at ends of chains situated further toward the core of the dendritic polymer.

Furthermore, when they are present, the bifunctional monomers (II) are spacing elements in the three-dimensional structure. They make it possible to control the branching density.

When they are present, the monomers (III) form nuclei. The “chain-limiting” monofunctional monomers (IV) are situated at the periphery of the dendrimers. It is specified that the fact that the periphery is covered with monofunctional monomers (IV) does not rule out the possibility of monofunctional monomers (IV) being present at ends of chains situated further toward the core of the dendritic polymer.

The presence of monomers (III) and (IV) makes it possible in particular to control the molecular weight.

Preferably, the dendritic polymers employed according to the invention are hyperbranched polyamides; they are obtained from at least one monomer of formula (I) exhibiting, as reactive polycondensation functional groups, amino functional groups and carboxyl antagonistic functional groups or from a monomer composition comprising in addition at least one monomer of formula (II) and/or (III) and/or (IV) exhibiting the same type(s) of reactive polycondensation functional group(s), it being possible for all or part of the monomer or monomers of formula (II) to be replaced by a lactam.

The polycondensation/polymerization operation can be carried out in a known way in the molten or solvent phase, it being possible for the monomer of formula (II), when it is present, to favourably act as solvent.

The operation can favourably be carried out in the presence of at least one polycondensation catalyst and optionally of at least one antioxidant. Such catalysts and antioxidants are known to a person skilled in the art. Mention may be made, as examples of catalysts, of phosphorus compounds, such as phosphoric acid, phosphorous acid, hypophosphorous acid, phenylphosphonic acids, such as 2-(2′-pyridyl)ethylphosphonic acid, or phosphites, such as tris(2,4-di(tert-butyl)phenyl)phosphite. Mention may be made, as examples of antioxidants, of antioxidants with a doubly-hindered phenol base, such as N,N′-hexamethylenebis(3,5-di(tert-butyl)-4-hydroxyhydrocinnamamide) or 5-tert-butyl-4-hydroxy-2-methylphenyl sulfide.

Hyperbranched polyamides exhibiting hydrophilic functionalities which do not react with the A, A′, A″, B, B′ and B″ functional groups can be obtained by employing a monomer of formula (III) and/or (IV) exhibiting one or more polyoxyethylene groups (for example monomer of the family of the Jeffamine aminated polyoxyalkylenes) and/or a monomer of formula (IV) exhibiting quaternary ammonium (cationic), nitrile, sulfonate (anionic), phosphonate (anionic) or phosphate (anionic) functional groups.

Another embodiment consists, after preparing a hyperbranched polymer by polycondensation of nonfunctionalized monomers, in modifying the end functional groups of said hyperbranched polyamide by reaction with a compound exhibiting hydrophilic and/or ionic or potentially ionic functional groups. It can, for example, be a compound exhibiting a tertiary amine (potentially cationic), quaternary ammonium (cationic), nitrile, sulfonate (anionic), phosphonate (anionic) or phosphate (anionic) group or polyoxyethylene groups. The end functional groups can also be modified by simple reactions of acid/base type, the groups included at the ends of chains being completely or partially ionized. For example, end groups of carboxylic acid type (for example B, B′ and/or B″ groups) can be rendered anionic by addition of a base. End groups of amine type (for example B, B′ and/or B″ groups) can be rendered cationic by addition of an acid.

It should be noted that the functionalization can be complete or partial. It is preferably greater than 25% by number, with respect to all the free functional groups carried (B, B′, B″).

It should be noted that the possibility is not ruled out of carrying out a partial hydrophobic functionalization after the preparation of the dendritic polymer.

The weight-average molar mass of said dendritic polymers, in particular hyperbranched polyamides, can range from 500 to 1 000 000 g/mol, preferably from 1000 to 500 000 g/mol, more preferably still from 3000 to 20 000 g/mol.

The weight-average molar mass can be measured by size exclusion chromatography. The measurement is carried out in an eluent phase composed of 70% by volume of Millipore 18 megaohms water and of 30% by volume of methanol, comprising 0.1M of NaNO₃; it is adjusted to pH 10 ( 1/1000 25% NH₄OH).

The weight-average molar mass is established in a known way via light scattering values.

Anionic Surfactant (b)

The anionic surfactants can in particular be chosen from the following compounds:
alkyl ester sulfonates of formula R—CH(SO₃M)-COOR′, where R represents a C₈-C₂₀, preferably C₁₀-C₁₆, alkyl radical, R′ represents a C₁-C₆, preferably C₁-C₃, alkyl radical and M represents an alkali metal (sodium, potassium or lithium) cation, a substituted or unsubstituted ammonium (methyl-, dimethyl-, trimethyl- or tetramethylammonium, dimethylpiperidinium, and the like) cation or a cation derived from an alkanolamine (monoethanolamine, diethanolamine, triethanolamine, and the like). Mention may very particularly be made of methyl ester sulfonates in which the R radical is a C₁₄-C₁₆ radical;
alkyl sulfates of formula ROSO₃M, where R represents a C₅-C₂₄, preferably C₁₀-C₁₈, alkyl or hydroxyalkyl radical, M representing a hydrogen atom or a cation with the same definition as above, and their ethoxylenated (EO) and/or propoxylenated (PO) derivatives, having on average from 0.5 to 30, preferably from 0.5 to 10, EO and/or PO units;
sulfated alkylamides of formula RCONHR′OSO₃M, where R represents a C₂-C₂₂, preferably C₆-C₂₀, alkyl radical and R′ represents a C₂-C₃ alkyl radical, M representing a hydrogen atom or a cation with the same definition as above, and their ethoxylenated (EO) and/or propoxylenated (PO) derivatives, having on average from 0.5 to 60 EO and/or PO units;
salts of saturated or unsaturated C₈-C₂₄, preferably C₁₄-C₂₀, fatty acids,
C₉-C₂₀ alkylbenzenesulfonates,
primary or secondary C₈-C₂₂ alkylsulfonates,
alkylglycerolsulfonates,
the sulfonated polycarboxylic acids disclosed in GB-A-1 082 179,
paraffin sulfonates,
N-acyl-N-alkyltaurates,
alkyl phosphates,
isethionates,
alkylsuccinamates, alkylsulfosuccinates,
monoesters or diesters of sulfosuccinates,
N-acylsarcosinates,
alkylglycoside sulfates,
polyethoxycarboxylates; the cation being an alkali metal (sodium, potassium or lithium), a substituted or unsubstituted ammonium (methyl-, dimethyl-, trimethyl- or tetramethylammonium, dimethylpiperidinium, and the like) residue, or a residue derived from an alkanolamine (monoethanolamine, diethanolamine, triethanolamine, and the like);
their mixtures or combinations.
   Cationic Surfactant (b)

The cationic surfactants can be chosen in particular from salts of optionally polyethoxylated primary, secondary or tertiary fatty amines, quaternary ammonium salts, such as tetraalkylammonium, alkylamidoalkylammonium, trialkylbenzylammonium, trialkylhydroxyalkylammonium or alkylpyridinium chlorides or bromides, imidazoline derivatives, amine oxides possessing a cationic nature, their mixtures or combinations.

Amphoteric or Zwitterionic Surfactant (d)

These surfactants (true amphoteric surfactants comprising an ionic group and a potentially ionic group of opposite charge or zwitterionic surfactants simultaneously comprising two opposite charges) can be chosen in particular from the following surfactants:
betaines generally, in particular carboxybetaines, for example lauryl betaine (Mirataine BB from Rhodia) or octyl betaine; amidoalkyl betaines, such as cocamidopropyl betaine (CAPB) (Mirataine BDJ from Rhodia Chimie);
sulfobetaines or sultaines, such as cocamidopropyl hydroxysultaine (Mirataine CBS from Rhodia);
alkylamphoacetates and alkylamphodiacetates, such as, for example, comprising a coco or lauryl chain (Miranol C2M, C32 and L32 in particular from Rhodia);
alkylamphopropionates or alkylamphodipropionates (Miranol C2M SF);
alkyl amphohydroxypropyl sultaines (Miranol CS),
their mixtures or combinations.
   Neutral Surfactant (d)

These surfactants can be chosen in particular from the following surfactants:
alkoxylated fatty alcohols;
alkoxylated triglycerides;
alkoxylated fatty acids;
alkoxylated sorbitan esters;
alkoxylated fatty amines;
alkoxylated di(1-phenylethyl)phenols;
alkoxylated tri(1-phenylethyl)phenols;
alkoxylated alkylphenols;
the products resulting from the condensation of ethylene oxide with a hydroprobic compound resulting from the condensation of propylene oxide with propylene glycol, such as the Pluronic products sold by BASF;
the products resulting from the condensation of ethylene oxide with the compound resulting from the condensation of propylene oxide with ethylenediamine, such as the Tetronic products sold by BASF;
alkylpolyglycosides, such as those disclosed in U.S. Pat. No. 4,565,647;
fatty acid amides, for example C₈-C₂₀ fatty acid amides;
their mixtures or combinations.
   Polyanionic or Polycationic Polymer (c)

The polymer (c) is a polymer which comprises several units carrying a cationic or anionic charge. The polymer (c) can be a synthetic polymer or a polymer derived from a natural polymer. Such polymers are known to a person skilled in the art. They are also sometimes referred to as “polyelectrolytes”.

It should be noted that the polyanionic or polycationic nature of a polymer can depend on the pH (the polymer can be potentially polyanionic or polycationic depending on the pH of the aqueous phase). Thus, the pH of the composition and the polymer are such that the latter is polyanionic or polycationic. For simplicity, for the description and the definition of the polymers of the composition, a polymer comprising anionic units or potentially anionic units in the neutral form is regarded as “polyanionic” or a polymer comprising cationic units or potentially cationic units in the neutral form is regarded as “polycationic”.

The polymer (c) is advantageously water-soluble or water-dispersible.

Polymer (c) of Natural Origin:

Mention may in particular be made of cationic derivatives of cellulose and cationic derivatives of guar gums which are optionally hydroxyalkylated.

Mention may be made, among the preferred cationic polymers, of cationic guar hydroxyalkylated (C₂-C₂₂) derivatives, such as, in particular, hydroxypropyl guar hydroxypropyltrimonium chloride (Jaguar C162, Jaguar C2000 and Jaguar C1000 sold by Rhodia), and cationic derivatives of cellulose, such as, in particular, the 3-(trimethylammonio)propylcellulose chloride 2-hydroxypoly(oxyethane-1,2-diyl) ether or polyquaternium-10 (Polymer JR400 sold by Union Carbide). Guar hydroxypropyltrimonium chlorides can also be used, for example Jaguar C-13S, C-14S, C-17 or Excel, all sold by Rhodia.

The cationicity of these polymers is variable; thus, in the case of hydroxypropylated derivatives of cationic guar gum, such as Jaguar C162 and Jaguar C2000 sold by Rhodia, the degree of hydroxypropylation (molar substitution or MS) will be between 0.02 and 1.2 and the degree of cationicity (degree of substitution or DS) will be between 0.01 and 0.6. These products can optionally be functionalized by hydrophobic groups, such as alkyl chains.

These cationic polymers can optionally be functionalized by anionic groups, such as carboxymethyl, sulfate, sulfonate or phosphate groups, provided that the degree of substitution of these anionic groups is in all cases less than the degree of substitution of the cationic groups.

Use may also be made of cationic cellulose derivatives, such as a cellulose 2-(2-hydroxy-3-(trimethyl-ammonio)propoxy)ethyl ether chloride, or polyquaternium-10 (JR400 polymer sold by Union Carbide).

These cationic polymers (a′) generally exhibit a molecular weight of at least 2000, more generally of the order of 200 000 to 3 000 000.

Synthetic Polymer (c)

The polymer (c) can be a synthetic polymer comprising cationic units (including potentially cationic units) and/or anionic units (including potentially anionic units). It can be a homopolymer, it can also be a copolymer comprising at least two different units (for example cationic or anionic units and neutral units). It can be a polymer or copolymer anionized or cationized by treatment subsequent to polymerization.

Advantageous polymers are cationic (co)polymers (including potentially cationic (co)polymers) comprising units deriving from cationic monomers (including potentially cationic monomers), optionally units derived from anionic monomers (including potentially anionic monomers) and optionally neutral units deriving from neutral monomers (hydrophilic and/or hydrophobic neutral monomers).

Advantageous polymers are anionic (co)polymers (including potentially anionic (co)polymers) comprising units deriving from anionic monomers (including potentially anionic monomers), optionally units derived from cationic monomers (including potentially cationic monomers) and optionally neutral units deriving from neutral monomers (hydrophilic and/or hydrophobic neutral monomers).

The polymer (c) is advantageously water-soluble or water-dispersible. If it comprises neutral units, the latter are advantageously hydrophilic. However, the possibility is not ruled out of it comprising hydrophobic neutral units.

Such polymers, copolymers, units, monomers and processes are known to a person skilled in the art.

Mention may be made, as examples of useful monomers, of the monomers below.

Mention may be made, as examples of potentially cationic monomers, of:
ω-(N,N-dialkylamino)alkylamides of α,β-mono-ethylenically unsaturated carboxylic acids, such as N,N-dimethylaminomethylacrylamide or -methacrylamide, 2-(N,N-dimethylamino)ethyl-acrylamide or -methacrylamide, 3-(N,N-dimethyl-amino)propylacrylamide or -methacrylamide, or 4-(N,N-dimethylamino)butylacrylamide or -methacrylamide
α,β-monoethylenically unsaturated aminoesters, such as 2-(dimethylamino)ethyl acrylate (DMAA), 2-(dimethylamino)ethyl methacrylate (DMAM), 3-(dimethylamino)propyl methacrylate, 2-(tert-butyl-amino)ethyl methacrylate, 2-(dipentylamino)ethyl methacrylate or 2-(diethylamino)ethyl methacrylate
vinylpyridines
vinylamine
vinylimidazolines
monomers which are precursors of amine functional groups, such as N-vinylformamide, N-vinylacetamide, and like, which generate primary amine functional groups by simple acidic or basic hydrolysis.

Mention may be made, as examples of cationic monomers, of:
ammonium acryloyl or acryoyloxy monomers, such as
   trimethylammoniopropyl methacrylate chloride,
   trimethylammonioethylacrylamide or -meth-acrylamide chloride or bromide,
   trimethylammoniobutylacrylamide or -meth-acrylamide methyl sulfate,
   trimethylammoniopropylmethacrylamide methyl sulfate (MES),
   (3-methacrylamidopropyl)trimethylammonium chloride (MAPTAC),
   (3-acrylamidopropyl)trimethylammonium chloride (APTAC),
   methacryloyloxyethyltrimethylammonium chloride or methyl sulfate,
   acryloyloxyethyltrimethylammonium chloride;
1-ethyl-2-vinylpyridinium or 1-ethyl-4-vinyl-pyridinium bromide, chloride or methyl sulfate;
N,N-dialkyldiallylamine monomers, such as N,N-di-methyldiallylammonium chloride (DADMAC);
polyquaternary monomers, such as dimethylamino-propylmethacrylamide, N-(3-chloro-2-hydroxypropyl), trimethylammonium chloride (DIQUAT), and the like.

Mention may be made, as examples of anionic or potentially anionic monomers, of:
monomers having at least one carboxyl functional group, such as α,β-ethylenically unsaturated carboxylic acids or the corresponding anhydrides, such as acrylic acid, methacrylic acid, maleic acid, acrylic anhydride, methacrylic anhydride, maleic anhydride, fumaric acid, itaconic acid, N-methacryloylalanine, N-acryloylglycine and their water-soluble salts
monomers which are precursors of carboxylate functional groups, such as tert-butyl acrylate, which bring about, after polymerization, carboxyl functional groups by hydrolysis
monomers having at least one sulfate or sulfonate functional group, such as 2-sulfooxyethyl methacrylate, vinylbenzenesulfonic acid, allylsulfonic acid, 2-acylamido-2-methylpropane-sulfonic acid, sulfoethyl acrylate or methacrylate, sulfopropyl acrylate or methacrylate, and their water-soluble salts
monomers having at least one phosphonate or phosphate functional group, such as vinylphosphonic acid, and the like, ethylenically unsaturated esters of phosphates, such as the phosphates derived from hydroxyethyl methacrylate (Empicryl 6835 from Rhodia) and those derived from polyoxyalkylene methacrylates, and their water-soluble salts.

Mention may be made, as examples of neutral hydrophilic monomers, of:
hydroxyalkyl esters of α,β-ethylenically unsaturated acids, such as hydroxyethyl acrylate, hydroxypropyl acrylate, hydroxyethyl methacrylate, hydroxypropyl methacrylate, glycerol monomethacrylate, and the like
α,β-ethylenically unsaturated amides, such as acrylamide, N,N-dimethylmethacrylamide, N-methylol-acrylamide, and the like
α,β-ethylenically unsaturated monomers carrying a water-soluble polyoxyalkylenated segment of the poly(ethylene oxide) type, such as poly(ethylene oxide) α-methacrylates (Bisomer S20W, S10W, and the like, from Laporte) or α,ω-dimethacrylates, Sipomer BEM from Rhodia (ω-behenyl polyoxyethylene methacrylate), Sipomer SEM-25 from Rhodia (ω-tristyrylphenyl polyoxyethylene methacrylate), and the like
α,β-ethylenically unsaturated monomers which are precursors of hydrophilic units or segments, such as vinyl acetate, which, once polymerized, can be hydrolyzed to produce vinyl alcohol units or poly(vinyl alcohol) segments
vinylpyrrolidones
α,β-ethylenically unsaturated monomers of ureido type and in particular methacrylamido 2-imidazolidinone ethyl (Sipomer WAM II from Rhodia).

Mention may be made, as examples of hydrophobic monomers, of:
vinylaromatic monomers such as styrene, α-methyl-styrene, vinyltoluene, and the like
vinyl or vinylidene halides, such as vinyl chloride or vinylidene chloride
C₁-C₁₂ alkyl esters of α,β-monoethylenically unsaturated acids, such as methyl acrylate, methyl methacrylate, ethyl acrylate, ethyl methacrylate, butyl acrylate, butyl methacrylate, 2-ethylhexyl acrylate, and the like
vinyl or allyl esters of saturated carboxylic acids, such as vinyl or allyl acetates, propionates, versatates, stearates, and the like
α,β-monoethylenically unsaturated nitriles comprising from 3 to 12 carbon atoms, such as acrylonitrile,. methacrylonitrile, and the like
α-olefins, such as ethylene, and the like
conjugated dienes, such butadiene, isoprene or chloroprene,
monomers capable of generating polydimethylsiloxane (PDMS) chains.

Mention may be made of the synthetic cationic polymers commonly used as conditioners in the field of cosmetics, such as polyquaternium 2, 6, 7 or 11 (INCI name), or such as polymethacrylamidopropyltrimonium chloride, for example sold by Rhodia under the name Polycare 133, or such as copolymers of DADMAC, of acrylic acid and optionally of acrylamide, sold under the Merquat range by Nalco.

Particles (e)

The composition can comprise solid or liquid particles of water-insoluble organic or inorganic compounds (e). In the case of liquid particles, it is also possible to use the term “emulsion”.

Said particles can be present in said compositions in a proportion of the order of 0.1 to 10% by weight, preferably of the order of 0.2 to 2% by weight. Their size can be between 0.15 and 70 microns.

Mention may be made, among the insoluble organic compounds which can be present in the form of particles in aqueous dispersion in said compositions, of water-insoluble and nonvolatile organopolysiloxanes (also referred to subsequently as “water-insoluble and nonvolatile silicones”), among which may be mentioned polyalkylsiloxane, polyarylsiloxane or polyalkylaryl-siloxane oils, gums or resins, or their water-insoluble functionalized derivatives, or their mixtures, which are nonvolatile.

Said organopolysiloxanes are regarded as water-insoluble and nonvolatile when their solubility in water is less than 50 g/liter and their intrinsic viscosity is at least 3000 mPa·s at 25° C.

Mention may be made, as more specific examples of water-insoluble and nonvolatile organopolysiloxanes or silicones, of silicone gums, such as, for example, diphenyl dimethicone gum, sold by Rhodia, and preferably polydimethylsiloxanes exhibiting a viscosity at least equal to 600 000 mPa·s at 25° C. and more preferably still those with a viscosity of greater than 2 000 000 mPa·s at 25° C., such as Mirasil DM 500000, sold by Rhodia.

The water-insoluble and nonvolatile organopolysiloxane or silicone occurs in the disperse form in the cosmetic composition comprising it. The water-insoluble and nonvolatile organopolysiloxane or silicone exists in the form of particles, the size of which can be chosen according to the nature of the cosmetic composition or the performance desired for said composition. Generally, this size can be from 0.02 to 70 microns. Preferably, this size is of the order of 1 to 80 microns, very particularly of the order of 1 to 30 microns.

Mention may also be made, as insoluble organic compounds (e) which can be present in the form of particles, of oils which may perform conditioning, protective or emollient roles, oils generally chosen from alkyl monoglycerides, alkyl diglycerides, triglycerides, such as oils extracted from plants (palm oil, coconut oil, cottonseed oil, soybean oil, sunflower oil, olive oil, grape seed oil, sesame oil, groundnut oil, castor oil, and the like) or oils of animal origin (tallow, fish oils, and the like), derivatives of these oils, such as hydrogenated oils, lanolin derivatives, mineral oils or liquid paraffins, perhydrosqualane, squalene, diols, such as 1,2-dodecanediol, cetyl alcohol, stearyl alcohol, oleyl alcohol, fatty esters, such as isopropyl palmitate, 2-ethylhexyl cocoate, myristyl myristate, lactic acid esters, stearic acid, behenic acid or isostearic acid.

Mention may also be made of particles of bactericidal or fungicidal agents in order to improve the disinfecting of the skin, such as, for example, triclosan, antidandruff agents, such as zinc pyrithione or octopirox, or insecticidal agents, such as natural or synthetic pyrethroids. These different organic molecules can, if appropriate, be encapsulated beforehand in appropriate matrices according to methods known to a person skilled in the art. Mention may be made, among these, by way of example, of the encapsulation of organic molecules in polymer latices.

It should be mentioned that the solid or liquid particles can be stabilized in the composition using agents such as emulsifying agents or dispersing agents.

Other Ingredients

The composition can comprise other ingredients to those mentioned above, for example in the monophase aqueous phase.

Use may obviously be made, in the composition, of acidic or basic pH regulating agents, for example citric acid or sodium, potassium or ammonium hydroxide.

The composition can comprise salts, for example sodium or potassium chloride.

Mention should be made, for example, of sequestering agents, softening agents, foam modifiers, colorants, pearlescence agents (pearlizers), moisturizing agents, antidandruff or antiseborrhic agents, suspending agents, emulsifying agents, ceramides, pseudoceramides, electrolytes, fatty acids, fatty acid esters, hydroxyl acids, thickeners, fragrances, preservatives, organic or inorganic sunscreens, proteins, vitamins, polymers or silicones. Some of these compounds are described in detail below.

The cosmetic compositions for hair and/or skin treatments can in particular comprise:
fixative resins, for example chosen from methyl acrylate/acrylamide copolymers, poly(vinyl methyl ether)/maleic anhydride copolymers, vinyl acetate/crotonic acid copolymers, octylacrylamide/methyl acrylate/butylaminoethyl methacrylate copolymers, polyvinylpyrrolidones, polyvinylpyrrolidone/methyl methacrylate copolymers, polyvinylpyrrolidone/vinyl acetate copolymers, poly(vinyl alcohol)s, poly(vinyl alcohol)/crotonic acid copolymers, poly(vinyl alcohol)/maleic anhydride copolymers, hydroxylpropyl celluloses, hydroxypropyl guars, sodium polystyrenesulfonates, polyvinylpyrrolidone/ethyl methacrylate/methacrylic acid terpolymers, monomethyl ethers of poly(methyl vinyl ether/maleic acid), poly(vinyl acetate)s grafted to polyoxyethylene backbones (EP-A-219 048), or copolyesters derived from terephthalic and/or isophthalic and/or sulfoisophthalic acid, anhydride or a diester thereof and from a diol. Preferably, the fixative resins are of the following type: polyvinylpyrrolidone (PVP), copolymers of polyvinylpyrrolidone and of methyl methacrylate, copolymers of polyvinylpyrrolidone and of vinyl acetate (VA), poly(ethylene glycol terephthalate)/poly(ethylene glycol) copolymers, poly(ethylene glycol terephthalate)/poly(ethylene glycol)/poly(sodium sulfoisophthalate) copolymers, and their blends.
polymeric derivatives performing a protective role, for example in amounts of the order. of 0.01-10%, preferably approximately 0.1-5% and very particularly of the order of 0.2-3% by weight, for example cellulose derivatives, such as cellulose hydroxyethers, methylcellulose, ethylcellulose, hydroxypropyl methylcellulose or hydroxybutyl methylcellulose, or poly(vinyl ester)s grafted to polyalkylene backbones, such as poly(vinyl acetate)s grafted to polyoxyethylene backbones (EP-A-219 048), or poly(vinyl alcohol)s.
plasticizers, for example from 0.1 to 10% of the formulation, preferably from 1 to 10%, for example adipates, phthalates, isophthalates, azelates, stearates, silicone copolyols, glycols, castor oil, or their mixtures.
metal-sequestering agents, more particularly those sequestering calcium, such as citrate ions.
humectants; mention may be made of glycerol, sorbitol, urea, collagen, gelatin, aloe vera or hyaluronic acid.
to further reduce irritation of or attack on the scalp, water-soluble or water-dispersible polymers, such as collagen or some non-allergizing derivatives of animal or plant proteins (wheat protein hydrolysates, for example), natural hydrocolloids (guar gum, locust bean gum, tara gum, and the like) or hydrocolloids resulting from fermentation processes, and derivatives of these polycarbohydrates, such as modified celluloses (for example, hydroxyethylcellulose or carboxymethylcellulose), or guar or locust bean derivatives, such as their nonionic derivatives (for example, hydroxypropylguar) or anionic derivatives (carboxymethylguar and carboxymethylhydroxypropylguar).
preservatives, such as the methyl, ethyl, propyl and butyl esters of p-hydroxybenzoic acid, sodium benzoate, germaben (trade name) or any chemical agent which prevents bacterial proliferation or molds and which is conventionally used in cosmetic compositions, are generally introduced into these compositions at a level of 0.01 to 3% by weight. The amount of these products is generally adjusted in order to prevent any proliferation of bacteria, molds or yeasts in the cosmetic compositions.
agents which modify the activity of the water and which greatly increase the osmotic pressure, such as carbohydrates or salts.
fragrances.
opacifying agents, such as pigments.
viscosifying or gelling polymers, such as crosslinked polyacrylates, cellulose derivatives, such as hydroxyl-propylcellulose or carboxymethylcellulose, guar gum and their derivatives, and the like, used alone or in combination, or the same compounds, generally in the form of water-soluble polymers modified by hydrophobic groups covalently bonded to the polymer backbone, as described in patent WO 92/16187, and/or water, in order to bring the total of the constituents of the formulation to 100%.
polymeric dispersing agents in an amount of the order of 0.1-7% by weight, in order to control the calcium and magnesium hardness, agents such as:
   water-soluble salts of polycarboxylic acids with a molecular weight of the order of 2000 to 100 000, obtained by polymerization or copolymerization of ethylenically unsaturated carboxylic acids, such as acrylic acid, maleic acid, maleic anhydride, fumaric acid, itaconic acid, aconitic acid, mesaconic acid, citraconic acid or methylenemalonic acid, and very particularly polyacrylates with a molecular weight of the order of 2000 to 10 000 (U.S. Pat. No. 3,308,067) or copolymers of acrylic acid and of maleic anhydride with a molecular weight of the order of 5000 to 75 000 (EP-A-66 915).
   poly(ethylene glycol)s with a molecular weight of the order of 1000 to 50 000.

The compositions for the treatments of hard surfaces can comprise in particular:
polymeric thickeners
hydrophilizing polymers
soil release polymers
antifoaming agents
foaming agents
agents which stabilize foams or which stimulate foams
fragrances or scents
agents for controlling the pH and/or the hardness of the water
salts, fillers
chelating agents
colorants
preservatives
enzymes
corrosion inhibitors
scale inhibitors
dyes
fluorescent whitening agents
agents
solvents
opacifying agents.
   Treatments of Surfaces—Applications

The invention also relates to a process for the treatment of a surface comprising the following stages:
a) application of the composition, and
b) optionally rinsing.

During this process, advantageously at least one of the compounds chosen from (a), (b), (c), if it is present, and (e), if it is present, is deposited at the surface. Without wishing to be committed to any one theory, it is believed that the deposition can be induced by the rinsing, which is a dilution, and/or by a change in pH and/or by simple affinity for the surface.

The surface can be a hair and/or the skin. In this case, the composition is a cosmetic composition, such as a shampoo intended to be rinsed off, a shower gel intended to be rinsed off, a conditioner intended to be rinsed off or a conditioner not intended to be rinsed off. The composition or the process can thus condition, repair or protect the hair and/or dye the hair, as mentioned above. The skin and/or the hair may have been wetted beforehand.

In the case of a shampoo, for example, the dilution factor of the cosmetic composition during its application can be evaluated at a value of the order of 3 to 10, it being considered that one gram of wet hair retains on average from 0.6 to 1 g of water and that, in a standard way, 0.1 g of shampoo is applied per gram of hair, expressed on a dry basis, with a duration of application generally of 30 to 45 seconds. After having been applied, the shampoo should then be rinsed off in order to remove the excess surface-active agents. According to an alternative form of the process of the invention, the composition is applied to the unwetted hair or skin, then the skin or hair is wetted and, finally, rinsed.

The surface can be a hard surface. In this case, the composition is a composition for cleaning (detergent composition) and/or treating hard surfaces, preferably domestic surfaces (including the dishes) or institutional or industrial surfaces. It should be noted that the composition can be applied to the hard surface by any means, optionally after being diluted beforehand. The composition can in particular be supported by a fibrous or porous product, such as wipes, pads, sponges, and the like.

Thus, the composition can be:
a dishwashing liquid, preferably for washing dishes by hand,
a composition for cleaning glass panes, in particular window panes and motor vehicle windshields,
a composition for washing floors,
a “universal” washing composition for any surface,
a composition for cleaning kitchen and/or bathroom surfaces,
a composition for cleaning toilets
a composition for cleaning showers, for the prevention or the formation of marks in showers (shower rinse or daily shower),
a composition for cleaning cars,
a composition for cleaning tiles or plastic floor coverings.

The treated surface can thus be:
glass, for example in windows and windshields,
tiles or ceramics, for example in kitchens, bathrooms, toilets, showers, dishes or floors,
metal, for example in dishes, motor vehicle bodies, window frames or floors,
plastic, for example in dishes, motor vehicles, windows, furniture or floors,
cement or concrete, optionally wax, for example in floors.

Other details or advantages of the invention will become more clearly apparent in the light of the examples which follow, without a limiting nature.

### EXAMPLES

### Example 1

Synthesis of a hyperbranched copolyamide comprising tertiary amine endings by melt copolycondensation of benzene-1,3,5-tricarboxylic acid (recorded as BTC, core molecule of R¹—B″₃ type, with B″═COOH), of 5 aminoisophthalic acid (recorded as AIPA, branching molecule of A-R—B₂ type, with A=NH₂ and B═COOH), of ε-caprolactam (recorded as CL, spacer of A′-R′—B′ type, with A′=NH₂ and B′═COOH) and of N-(3-aminopropyl)morpholine (recorded as APM, blocker of A″-R² type, with A″=NH₂). The respective overall composition is 1/6/12/9 BTC/AIPA/CL/APM.

The reaction is carried out in a 500 ml glass reactor commonly used in the laboratory for the melt synthesis of polyesters or polyamides.

A Wood's alloy metal bath is employed for the heating of the reaction mixture.

88.1 g of N-(3-aminopropyl)morpholine (0.611 mol) and 92.2 g of ε-caprolactam (0.815 mol) are introduced into the reactor at ambient temperature. The reactor is then heated to 100° C. and stirred mechanically.

Once the temperature is reached, 73.8 g of 5-amino-isophthalic acid (0.407 mol) and 14.3 g of benzene-1,3,5-tricarboxylic acid (0.068 mol) are added. Once the reaction mixture is homogeneous, 0.605 g of a 50% (w/w) aqueous hypophosphorous acid solution is added. Gentle flushing with dry nitrogen is subsequently carried out.

The reaction mass is then rapidly heated from 100° C. to 160° C. over 12 min. After an isothermal stationary phase lasting 120 min, the temperature is increased to 200° C. over approximately 80 min. After 60 minutes under these conditions, the temperature is again increased to 250° C. over approximately 10 min and then maintained under these conditions until the end of the synthesis.

After a stationary phase lasting 50 min, the reactor is gradually placed under vacuum over a period of approximately 60 min and then kept under maximum vacuum (24 mbar) for an additional hour.

At the end of the cycle, stirring is halted and the reactor is allowed to cool to ambient temperature under a stream of nitrogen. 236.8 g of polymer are collected. The hyperbranched copolyamide obtained is a yellowish solid which is soluble in an acidic aqueous phase.

The content of tertiary amine functional group is determined by direct potentiometric assay of a solution of hyperbranched copolyamide in a 30/70 by volume chloroform/trifluoroethanol mixture by 0.05N hydrochloric acid. The content of amine groups obtained is 2187 meq/kg.

### Example 2

Synthesis of hyperbranched copolyamide comprising tertiary amine endings by melt copolycondensation of BTC, of AIPA, of CL and of APM. The respective overall composition is 1/25/50/28 BTC/AIPA/CL/APM.

The same reactor is employed as that described in example 1. A Wood's alloy metal bath is employed for the heating of the reaction mixture.

74.9 g of N-(3-aminopropyl)morpholine (0.519 mol), 104.9 g of ε-caprolactam (0.927 mol), 84.0 g of 5-amino-isophthalic acid (0.464 mol) and 3.9 g of benzene-1,3,5-tricarboxylic acid (0.019 mol) are introduced into the reactor at ambient temperature. The reactor is subsequently placed under mechanical stirring under a gentle stream of dry nitrogen and is then heated to 100° C. Once the reaction mixture is homogeneous, 0.630 g of a 50% (w/w) aqueous hypophosphorous acid solution is added.

The reaction mass is heated according to the same cycle as that described in example 1. The final cycle under reduced pressure is also repeated.

At the end of the cycle, stirring is halted and the reactor is allowed to cool to ambient temperature under a stream of nitrogen. 236.0 g of polymer are collected. The hyperbranched copolyamide obtained is a yellowish solid which is soluble in an acidic aqueous phase.

The content of amine groups, determined by potentiometric assay, is 2025 meq/kg.

### Example 3

Synthesis of hyperbranched copolyamide comprising tertiary amine endings by melt copolycondensation of BTC, of AIPA, of CL and of APM. The respective overall composition is 1/50/100/53 BTC/AIPA/CL/APM.

The same reactor is employed as that described in example 1. A Wood's alloy metal bath is employed for the heating of the reaction mixture.

72.5 g of N-(3-aminopropyl)morpholine (0.502 mol), 107.3 g of ε-caprolactam (0.948 mol), 85.9 g of 5-amino-isophthalic acid (0.474 mol) and 2.0 g of benzene-1,3,5-tricarboxylic acid (0.009 mol) are introduced into the reactor at ambient temperature. The reactor is subsequently placed under mechanical stirring under a gentle stream of dry nitrogen and is then heated to 100° C. Once the reaction mixture is homogeneous, 0.635 g of a 50% (w/w) aqueous hypophosphorous acid solution is added.

The reaction mass is heated according to the same cycle as that described in example 1. The final cycle under reduced pressure is also repeated.

At the end of the cycle, stirring is halted and the reactor is allowed to cool to ambient temperature under a stream of nitrogen. 236.20 g of polymer are collected. The hyperbranched copolyamide obtained is a yellowish solid which is soluble in an acidic aqueous phase.

The content of amine groups, determined by potentiometric assay, is 1963 meq/kg.

### Example 4

Quaternization by dimethyl sulfate of a hyperbranched copolyamide comprising tertiary amine endings with an overall composition of 1/6/12/9 respectively for BTC/AIPA/CL/APM synthesized in example 1.

The same glass reactor is employed as in examples 1 to 3, equipped with a vertical reflux condenser. An oil bath is employed as means for heating the reactor.

40.0 g (87.5 meq of amine) of hyperbranched copolyamide obtained in example 1 are finely milled and dispersed in 210.0 g of acetone. The mixture is stirred mechanically using an anchor stirrer and heated to reflux. 13.2 g of dimethyl sulfate (0.105 mol) are subsequently added over 5 min. The reaction mixture is maintained under these conditions for 4 hours. At the end of the reaction, the hyperbranched copolyamide exists in the form of a viscous gel. The reaction mixture is left to separate by settling and the supernatant acetone is removed.

The gel obtained is taken up to 210 g of demineralized water and heated at 100° C. for 30 min in order to destroy the traces of unreacted dimethyl sulfate. The solution is subsequently freeze dried.

The content of quaternized amine groups is determined by the potentiometric assay method described in example 1 and is 1984 meq/kg for this composition.

### Example 5

Quaternization by dimethyl sulfate of a hyperbranched copolyamide comprising tertiary amine endings with an overall composition of 1/25/50/28 respectively for BTC/AIPA/CL/APM synthesized in example 2.

The same arrangement is employed as that described in example 4.

40.0 g (81.0 meq of amine) of hyperbranched copolyamide obtained in example 2 are finely milled and dispersed in 210.0 g of acetone. The mixture is stirred mechanically using an anchor stirrer and heated to reflux. 12.3 g of dimethyl sulfate (0.097 mol) are added over 5 min when the mixture reaches 40° C. The reaction mixture is subsequently maintained by reflux of acetone for 4 hours. The viscous gel obtained is subsequently recovered after removal of the supernatant acetone, then taken in 250 g of demineralized water and heated at 100° C. for 30 min in order to destroy the traces of unreacted dimethyl sulfate. The solution is subsequently freeze dried.

The content of quaternized amine groups, determined by potentiometric assay, is 1735 meq/kg.

### Example 6

Quaternization by dimethyl sulfate of a hyperbranched copolyamide comprising tertiary amine endings with an overall composition of 1/50/100/53 respectively for BTC/AIPA/CL/APM synthesized in example 3.

The same arrangement is employed as that described in example 4.

40.0 g (78.5 meq of amine) of hyperbranched copolyamide obtained in example 3 are finely milled and dispersed in 210.0 g of acetone. The mixture is stirred mechanically using an anchor stirrer and heated to reflux. 11.9 g of dimethyl sulfate (0.094 mol) are added over 5 min when the mixture reaches 40° C. The reaction mixture is subsequently maintained by reflux of acetone for 4 hours. The viscous gel obtained is recovered after removal of the supernatant acetone, then taken in 250 g of demineralized water and heated at 100° C. for 30 min in order to destroy the traces of unreacted dimethyl sulfate. The solution is subsequently freeze dried.

The content of quaternized amine groups, determined by potentiometric assay, is 1688 meq/kg.

### Examples 7-9

Compositions comprising ingredients chosen from the following are prepared:

| Ingredient | Type | Compound |
|---|---|---|
| SLES | Anionic | Sodium lauryl ether sulfate (2 EO), |
| | surfactant | EMPICOL ESB/3M, sold by Huntsman |
| SLS | Anionic | Sodium lauryl sulfate |
| | surfactant | |
| ALES | Anionic | Ammonium lauryl ether sulfate (2 EO), |
| | surfactant | Rhodapex-EA-2, sold by Rhodia |
| ALS | Anionic | Ammonium lauryl sulfate, |
| | surfactant | Rhodapon L-22, sold by Rhodia |
| CAPB | Amphoteric | Cocamidopropyl betaine, |
| | surfactant | Mirataine BET-C-30, sold by Rhodia |
| Salt | | Sodium chloride or Ammonium chloride |
| Polymer A | | Polymer of example 4 |
| Polymer B | | Polymer of example 5 |
| Polymer C | | Polymer of example 6 |

Procedure
1. The water and the polymer are mixed.
2. The CAPB is added.
3. The anionic surfactant is added.
4. The pH is adjusted to 6-6.5 by addition of sodium hydroxide or citric acid
5. The salt is added.

The following compositions, the amount by weight of each ingredient of which is given below, are prepared:

| | Example | | |
|---|---|---|---|
| | 7 | 8 | 9 |
| SLES (%) | 14 | 14 | 14 |
| SLS (%) | / | / | / |
| ALES (%) | / | / | / |
| ALS (%) | / | / | / |
| CAPB (%) | 2 | 2 | 2 |
| NH₄Cl (%) | / | / | / |
| NaCl (%) | 1.5 | 1.5 | 1.5 |
| Polymer A (%) | 0.3 | 1 | / |
| Polymer B (%) | / | 0.3 | |
| Polymer C (%) | / | 7 | 0.3 |

| | |
|---|---|
| Water | Up to 100% |

The transmittance (transparency) of the compositions is measured at 600 nm using a spectrophotometer (Jasco 7800 type). The compositions exhibit a transparency of more than 90%.

The compositions facilitate combing with regard to wet hair (wet combing), in comparison with compositions not comprising the polymer.

1-13. (canceled)

14. A composition comprising:
a monophase aqueous phase comprising a system comprising:
water,
a water-soluble or water-dispersible polyionic dendritic polymer (a),
at least one ionic surfactant (b),
optionally a polycationic or polyanionic polymer (c) other than the dendritic polymer, and
optionally at least one amphoteric, zwitterionic or neutral surfactant (d),
in which:
(a) is polycationic, (b) is anionic and (c), if it is present, is polyanionic or polycationic, or
(a) is polycationic, (b) is cationic and (c) is present and polyanionic, or
(a) is polyanionic, (b) is cationic and (c), if it is present, is polyanionic or polycationic, or
(a) is polyanionic, (b) is anionic and (c) is present and polycationic,
the monophase aqueous phase becomes two-phase or remains monophase on diluting with water, and
optionally solid or liquid particles of water-insoluble organic or inorganic compounds (e).

15. The composition as claimed in claim 14, wherein (a), (b), (c), if it is present, and (d) if it is present, and their amounts are such that the monophase aqueous phase becomes two-phase on diluting with water.

16. The composition as claimed in claim 14, wherein:
the monophase aqueous phase becomes two-phase on diluting with water, and
(a) or (c) precipitates when the aqueous phase becomes two-phase on diluting with water.

17. The composition as claimed in claim 14, wherein the system comprises (c) and wherein
(a) is polycationic, (b) is anionic and (c) is polycationic, or
(a) is polyanionic, (b) is cationic and (c) is polyanionic.

18. The composition as claimed in claim 14, being an aqueous cosmetic composition for the hair and/or the skin intended to be rinsed off.

19. The composition as claimed in claim 18, being a shampoo, a conditioner or a shower gel.

20. The composition as claimed in claim 14, being a conditioner not intended to be rinsed off.

21. The composition as claimed in claim 14, being a composition for cleaning and/or treating hard surfaces.

22. A process for the treatment of a surface comprising the following stages:
a) application of a composition as claimed in claim 14, and
b) optionally rinsing.

23. The process as claimed in claim 22, wherein at least one of the compounds chosen from (a), (b), (c), if it is present, and (e), if it is present, is deposited at the surface.

24. The process as claimed in claim 23, wherein the surface is a hair and/or skin surface.

25. The process as claimed in claim 24, wherein the surface is a hard surface.

26. A process for the conditioning, repair or protection of the hair and/or in the dyeing of the hair comprising the step of treating said hair with a composition as defined in claim 19.
